(19) United States
(12) Patent Application Publication
(10) Pub. No.: US 2019/0247006 A1
(43) Pub. Date: Aug. 15, 2019

(54) CALIBRATION TARGET FOR QUANTITATIVE COMPUTED TOMOGRAPHY

(71) Applicant: CARESTREAM HEALTH, INC., Rochester, NY (US)

(72) Inventor: Lawrence A. Ray, Rochester, NY (US)

(21) Appl. No.: 15/908,873

(22) Filed: Mar. 1, 2018

Related U.S. Application Data
(60) Provisional application No. 62/629,221, filed on Feb. 12, 2018.

(51) Int. Cl. A61B 6/00; A61B 6/03

(52) U.S. Cl. CPC A61B 6/585; A61B 6/4085; A61B 6/035

(57) ABSTRACT

A wearable calibration target having a band that is configured to wrap about a patient's limb and one or more calibration patches coupled to the band, wherein each of the one or more calibration patches is formed from a material having a known attenuation to X-ray radiation.

### CROSS REFERENCE TO RELATED APPLICATIONS

This application claims the benefit of U.S. Provisional application U.S. Ser. No. 62/629,221, provisionally filed on Feb. 12, 2018, entitled “CALIBRATION TARGET FOR QUANTITATIVE COMPUTED TOMOGRAPHY”, in the name of Lawrence A. Ray, which is incorporated herein by reference in its entirety.

### FIELD OF THE INVENTION

The disclosure relates generally to diagnostic imaging and in particular to radiographic imaging systems used for obtaining volume images of patient extremities.

### BACKGROUND OF THE INVENTION

3-D volume imaging has proved to be a valuable diagnostic tool that offers significant advantages over earlier 2-D radiographic imaging techniques for evaluating the condition of internal structures and organs. 3-D imaging of a patient or other subject has been made possible by a number of advancements, including the development of high-speed imaging detectors, such as digital radiography (DR) detectors that enable multiple images to be taken in rapid succession.

Cone beam computed tomography (CBCT) or cone beam CT technology offers considerable promise as one type of diagnostic tool for providing 3-D volume images. Cone beam CT systems capture volumetric data sets by using a high frame rate digital radiography (DR) detector and an X-ray source, typically affixed to a gantry that rotates about the object to be imaged, directing, from various points along its orbit around the subject, a divergent cone beam of X-rays toward the subject. The CBCT system captures projections throughout the rotation, for example, one 2-D projection image at every degree of rotation. The projections are then reconstructed into a 3D volume image using various techniques. Among well known methods for reconstructing the 3-D volume image from the 2-D image data are filtered back projection and iterative algebraic reconstruction approaches.

Recent advances in CBCT offer improved capability for volume imaging of patient extremities, such as portions of the leg, arm, and shoulder, for example. A CBCT system for providing this function is described, for example, in commonly assigned U.S. Pat. No. 8,348,506 entitled “Extremity imaging apparatus for cone beam computed tomography” to Yorkston et al., incorporated herein by reference. Using this type of system, highly detailed volume images of the complex bone structures and joint arrangements characteristic of extremities can be obtained and analyzed as a useful diagnostic tool.

While CBCT has proved to be of valuable assistance for extremity diagnosis and treatment, however, there are some problems that constrain the overall accuracy of the information that is obtained. For example, one aspect of interest for extremity diagnosis and treatment and for bone condition overall relates to bone material density (BMD). Quantitative Computed Tomography (QCT) is a technique used to measure BMD. QCT obtains the attenuation data acquired for each bone voxel, expressed in Hounsfield Units (HU), and interprets this data as being linearly related to bone mineral density at that spatial location. Straightforward conversion of the HU data to BMD information can thus provide highly useful information to the diagnostician.

Obtaining accurate Hounsfield Unit data from the acquired image content, however, requires calibration. Most standard CT systems have a platform, e.g., a bed, used to move the patient into and through the scanning system. For stationary QCT systems there is often a set of calibration targets implanted into the bed or other platform in order to assure that the acquired data response of the radiography detector can be calibrated relative to objects of known HU values. Various alternative methods have been proposed for calibration during CT image acquisition, such as using a solid phantom placed beneath or against the patient, addressing the need for regular, ongoing calibration procedure without taking the imaging system out of service.

Methods suitable for CT system calibration, however, are not applicable to CBCT systems, particularly for portable systems and extremity imaging apparatus that are designed to adapt to variable patient location, limb orientation, and positioning. No bed or stationary platform is used; instead, the patient may be standing or sitting, according to the exam type, and may be instructed to extend the extremity of interest to an appropriate depth within the bore of an imaging system.

Executing or verifying calibration as a separate procedure before each patient imaging examination proves to be impractical, requiring considerable time, an experienced operator, and high expense. Moreover, as with computed tomography systems in general, maintaining ongoing accuracy of the CBCT imaging apparatus can be a problem due to spatial drift. With respect to Hounsfield units (HU), for some CBCT apparatus for example, there can be characteristic drift of HU values perceptible along the axial direction during scanning of patient anatomy. Change in system response can also be a consideration, with frequent re-calibration recommended to minimize drift between exams.

Thus, it can be seen that there would be significant benefit in a CBCT calibration solution that is suitable for extremity imaging.

### SUMMARY OF THE INVENTION

It is an object of the present disclosure to advance the art of diagnostic imaging and calibration for acquiring volume images of extremity body parts, particularly jointed or load-bearing, paired extremities such as knees, legs, ankles, fingers, hands, wrists, elbows, arms, and shoulders.

It is a feature of the present disclosure that it provides a mechanism for straightforward, automated calibration of the CBCT apparatus without requiring extensive operator training or procedures and without discomfort to the patient.

According to an aspect of the present disclosure there is provided a wearable calibration target comprising: a band that is configured to wrap about a patient's limb; and one or more calibration patches coupled to the band, wherein each of the one or more calibration patches is formed from a material having a known attenuation to X-ray radiation.

These objects are given only by way of illustrative example, and such objects may be exemplary of one or more embodiments of the disclosure. Other desirable objectives and advantages inherently achieved by the disclosure may occur or become apparent to those skilled in the art. The invention is defined by the appended claims.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a schematic view showing exemplary geometry for CBCT scanning for portions of the lower leg.

FIG. 2 is a perspective view showing an exemplary extremity CBCT volume imaging apparatus in simplified schematic form.

FIG. 3 is a top view that shows packaging of X-ray source and detector within a frame to allow movement about a scanned object.

FIG. 4 shows the CBCT frame in position for imaging of lower limbs.

FIG. 5 shows the CBCT frame in position for imaging of upper limbs.

FIG. 6 shows a wearable calibration target in the form of a band having calibration patches according to an embodiment of the present disclosure.

FIGS. 7 and 8 show a wearable target wrapped about the leg near an ankle that is the imaged object.

FIG. 9 shows the wearable target wrapped about the arm near the wrist of a patient.

FIG. 10 shows an elbow having two wearable targets spaced apart with respect to an axis of rotation.

### DETAILED DESCRIPTION OF THE INVENTION

The following is a detailed description of the preferred embodiments of the invention, reference being made to the drawings in which the same reference numerals identify the same elements of structure in each of the several figures.

In the context of the present disclosure, the term “extremity” has its meaning as conventionally understood in diagnostic imaging parlance, referring to knees, legs, ankles, fingers, hands, wrists, elbows, arms, and shoulders and any other anatomical extremity. The term “subject” is used to describe the extremity of the patient that is imaged, such as the “subject leg”, for example. The term “paired extremity” is used in general to refer to any anatomical extremity wherein normally two or more are present on the same patient. In the context of the present invention, the paired extremity is not imaged unless necessary; only the subject extremity is imaged.

To describe the present invention in detail, a number of the examples given herein for embodiments of the present invention focus on imaging of the load-bearing lower extremities of the human anatomy, such as the leg, the knee, the ankle, and the foot, for example. However, these examples are considered to be illustrative and non-limiting.

In the context of the present disclosure, the term “arc” or, alternately, “circular arc”, has its conventional meaning as being a portion of a circle of less than 360 degrees or, considered alternately, of less than 2π radians for a given radius.

The term “actuable” has its conventional meaning, relating to a device or component that is capable of effecting an action in response to a stimulus, such as in response to an electrical signal, for example.

As used herein, the term “energizable” relates to a device or set of components that perform an indicated function upon receiving power and, optionally, upon receiving an enabling signal.

An extremity imaging apparatus for Cone Beam Computed Tomography is described in WO 2014/058775 (Litzenberger) filed as PCT/US2013/063673, and in WO 2014/058771 (Litzenberger) filed as PCT/US2013/063666, both of which are incorporated herein in their entirety by reference. The imaging apparatus for cone beam computed tomography imaging of an extremity of a patient includes: a support structure that includes a support column; a vertical translation element for positioning in a height direction to a height position along the support column, and a scanner.

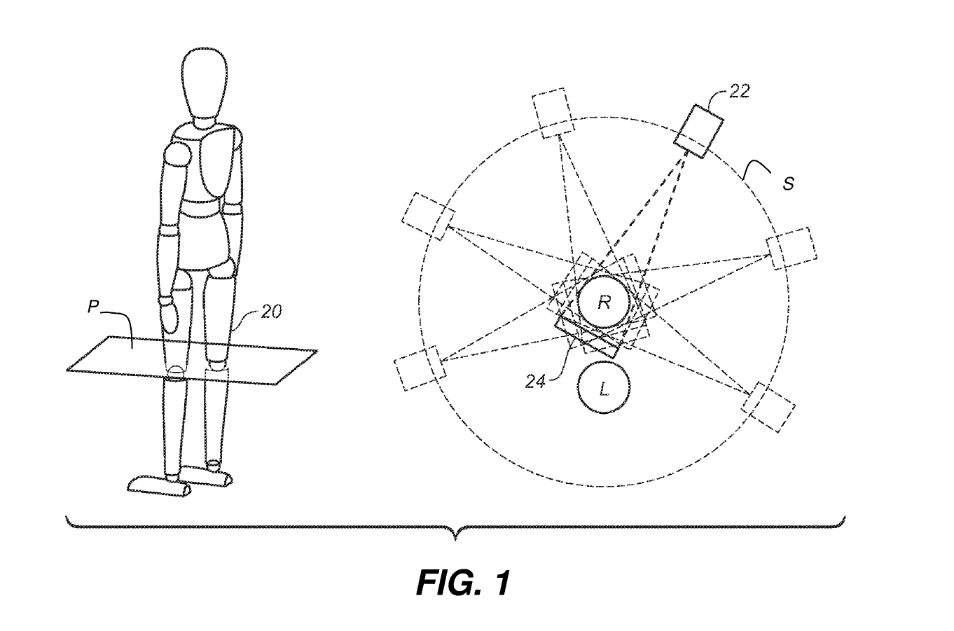

FIG. 1 shows perspective and top views of image capture geometry for extremity imaging of the right knee R of a patient as imaged object 20. In the top view, FIG. 1 shows the circular scan paths for a radiation source 22 and detector 24, which orbit along scan paths that lie within a plane P. A scan path S for source 22 is shown in the top view. Various angular positions of radiation source 22 and detector 24 along their orbit about subject 20 are shown in dashed line form. In practice, source 22, placed at some distance from the knee, can be positioned at different points over an arc of about 200 degrees; with any larger arc the paired extremity, left knee L, blocks the way. Detector 24, smaller than source 22 and typically placed very near subject 20, can be positioned between the patient's right and left knees and is thus capable of positioning over the full circular orbit.

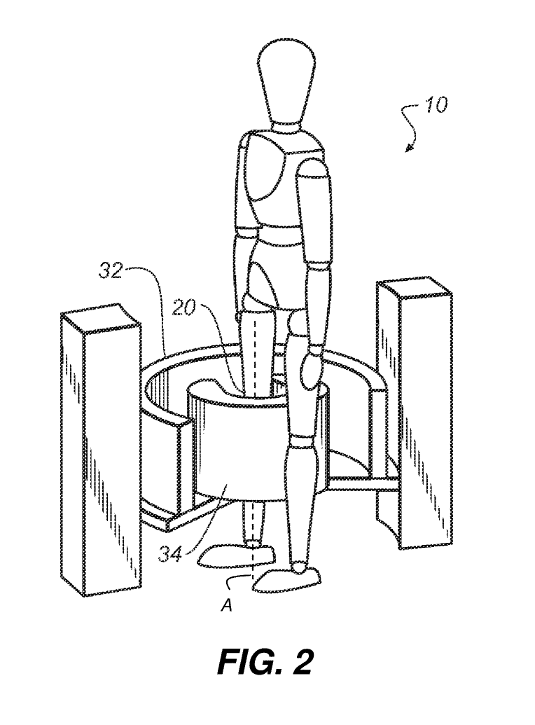

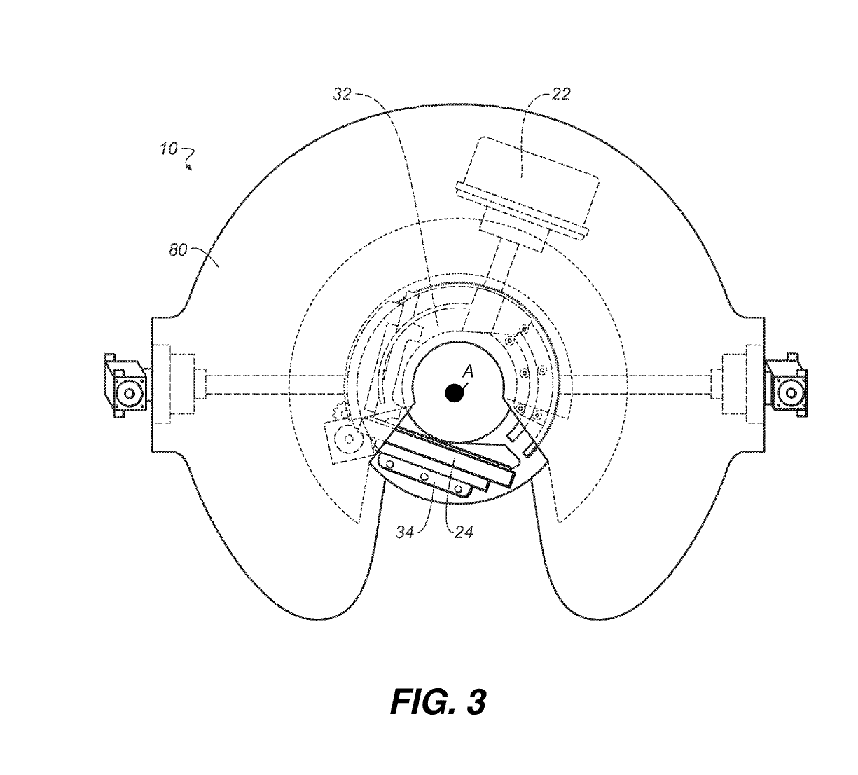

The perspective view of FIG. 2 shows an exemplary extremity CBCT volume imaging apparatus 10 in simplified schematic form. Subject 20, the patient's knee in this example, is positioned along an axis A of rotation, within a cavity that is within the scan path of the X-ray source, controlled by a source transport 32 and further within the scan path of the detector, controlled by a detector transport 34. The top view of FIG. 3 shows how source 22 and detector 34 can be packaged within a frame 80 that allows movement about the scanned subject.

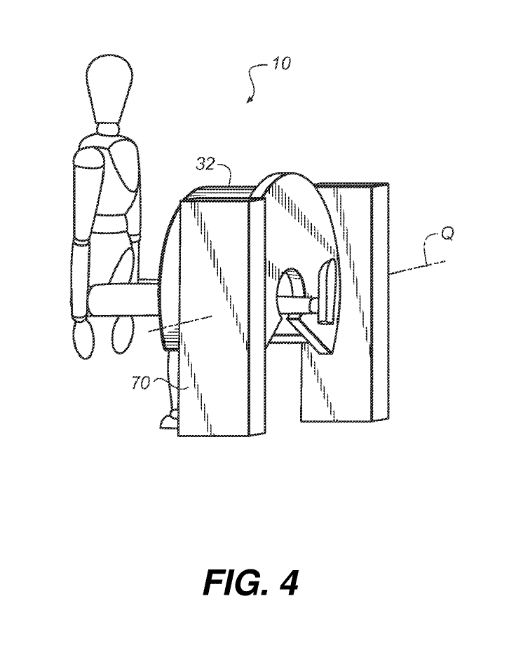

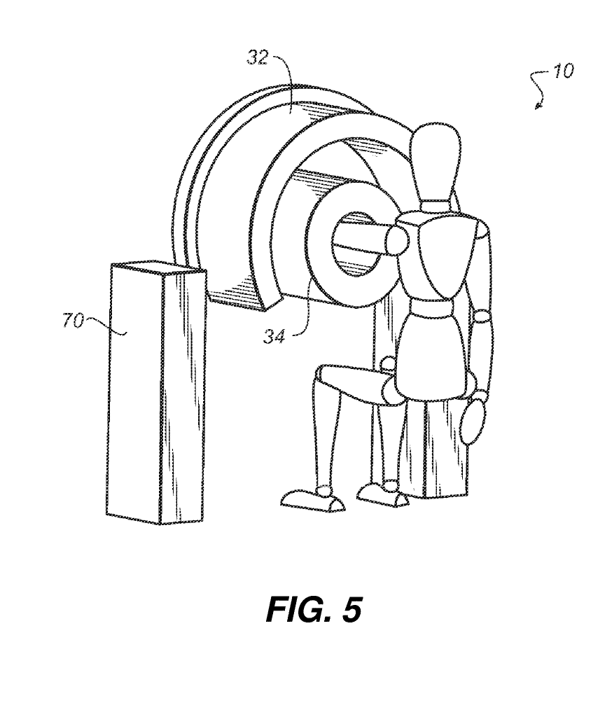

By way of illustrative example, FIGS. 4 and 5 show frame 80 in different angular positions for imaging of legs and arms or shoulders, respectively. Frame 80 can be rotatable, such as on an axis Q, to a suitable position and elevated to an appropriate height by a transport assembly 70.

An embodiment of the present disclosure addresses the need for providing a calibration target to facilitate automated calibration of a CBCT apparatus by providing one or more calibration targets in the form of a calibration target device. In a preferred arrangement, the calibration target device is a wearable device, such as in the form of a band or bracelet, that can be configured to be worn/wrapped about an arm, wrist, leg, or other limb or otherwise fitted around/adjacent/near the imaged extremity or configured to be attached to a piece of cloth/clothing fitted about the arm, wrist, leg, or limb.

The calibration target device can include one or more discrete/individual calibration target elements or patches of the same or different materials, arranged so that these target elements can be identified for processing in either the individual projection images or the reconstructed volume image.

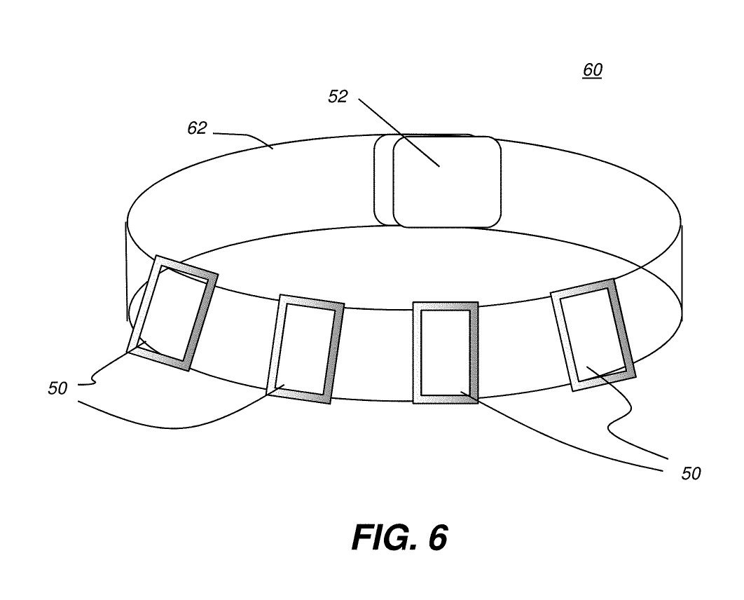

The perspective view of FIG. 6 shows an exemplary wearable calibration target device/system/apparatus 60 with one or more calibration target elements or patches 50. As illustrated in FIG. 6, calibration target device 60 is wearable in the form of a band/bracelet 62 configured to be worn/wrapped about an arm, wrist, leg, or other limb or otherwise fitted around the imaged extremity, or alternately, configured to be attached/coupled to a piece of cloth/clothing covering the imaged extremity.

Each calibration target patch 50 is formed from a suitable radio-opaque material having a known/predetermined size and exhibiting a known/predetermined attenuation to X-ray radiation, allowing straightforward computation of HU values. Patches 50 can be formed from materials of the same or different radiometric densities and any number of patches 50 can be used.

According to an embodiment of the present disclosure, patches 50 are formed from reference standards that are conventionally used in the CT calibration arts, formed of materials such as calcium hydroxyapatite, potassium phosphate, and distilled water. Other suitable radio-opaque materials for calibration, wherein the materials have known/predetermined attenuation to X-ray energy, could similarly be used.

The arrangement of calibration target elements/patches 50 can be configured according to the exam type. For example, particular practitioners may consider different patches 50 of particular materials to be more useful for imaging one type of limb than for imaging another type of limb.

Calibration target elements or patches 50 can be of any suitable shape and size, including plate-shaped, spherical, or other geometric shape. Shapes can relate to the material that is used in the patch; different materials could be provided as patches 50 having different shapes and colors. The patches 50 can have the corresponding reference material encased, such as in a radio-opaque plastic or other material. Patches 50 can be re-positioned around the band, shifted in position (i.e., slideably movable), and can be added to or removed from wearable target device 60 as needed. Patches 50 can be connected/coupled to a support 62 (illustrated as a band/bracelet) using any of a number of type of clips, clasps, snaps, fittings, adhesives, string or connective material, hook-and-loop fasteners, or other mountings/connectors/fasteners. As such, the calibration patches 50 can be removably attachable to band 62. That is, patch 50 can be attached and detached from band 62 without the destruction of patch 50. In such a manner, patches 50 of calibration target device 60 are disposed/positioned relative to a limb of a patient.

In one embodiment, patches 50 can dangle from portions of band 62, so that the calibration target elements are spaced apart from the patient's skin.

In an alternate embodiment, calibration target elements or patches 50 are built into the band 62, such as woven or sewed into the band, protected from contact. To promote sanitary conditions, band 62 can be comprised of an anti-bacterial material.

According to another alternate embodiment of the present disclosure, radiotransparent band 62 can extend over or around the joint or other extremity part to be imaged, covering the anatomy of interest with radiotransparent portions of the band and providing calibration target elements or patches 50 along outer edges of the band 62.

Band 62 can be a fixed circumferential size or can be adjustable to a number of discrete size settings. Band 62 can be stiff, such as a hinged ring or shell, or can be conformal and flexible, such as formed from a cloth or flexible synthetic material that conforms readily to the outer surface shape of the limb, fitting comfortably against the skin or clothing of the patient. Band 62 can be washable or disposable following removal of patches 50. Different sizes/colors of band 62 can be used for patients of different dimensions. Band 62 can be formed from a radio-transparent material.

Wearable target device 60 can be elastic and can have a fastener or clasp 52, such as a Velcro® brand hook-and-loop fastener, buckle, or other latching or fastening mechanism, such as a lacing mechanism, for example. Clasp 52 can allow adaptable sizing of band 62 (including its diameter) in order to tighten or loosen the band along/about the limb.

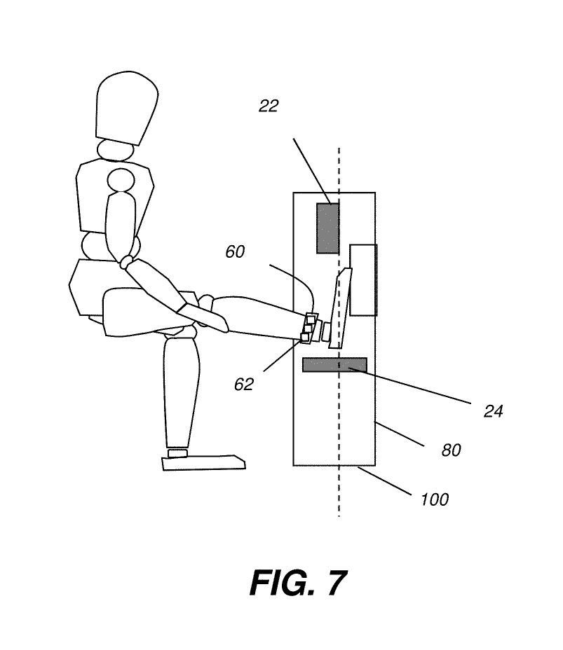

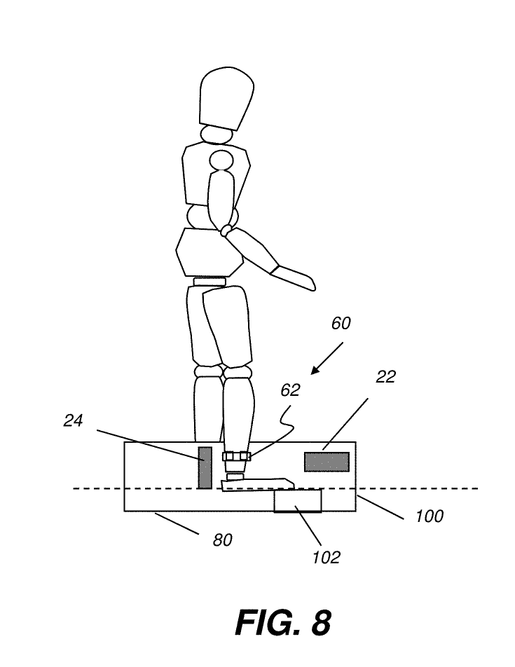

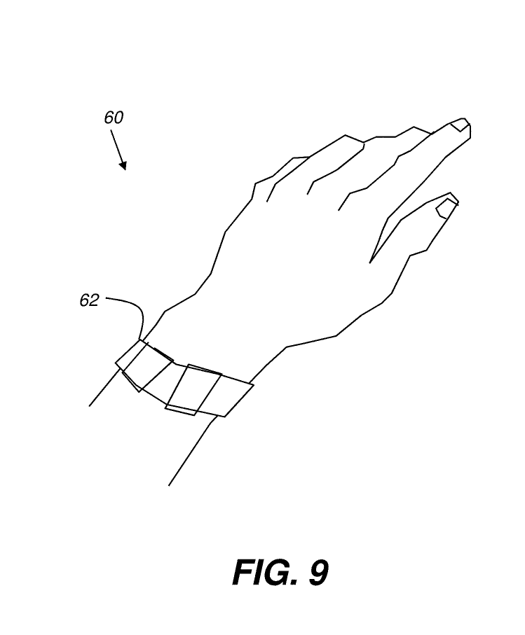

Wearable target device 60 can be worn on the limb or other extremity in any suitable position such that patches 50 are spaced apart from the imaged object. FIGS. 7 and 8 show band 62 wrapped about the leg near an ankle that is the imaged object in a volume imaging apparatus 100. The ankle may be elevated on a support 102. FIG. 9 shows band 62 wrapped about the arm near the wrist of a patient.

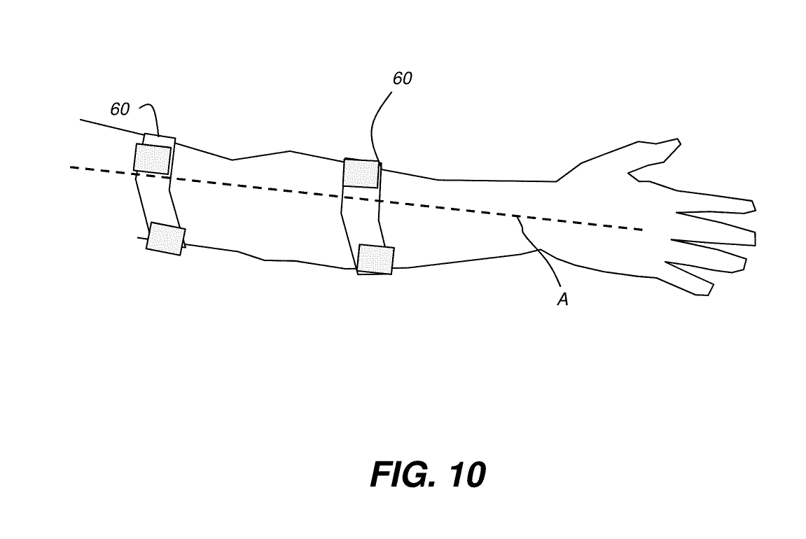

More than one wearable target device 60 can be used for a patient, allowing multiple patch or target elements 50 to be imaged, such as at different locations along the axial direction. The schematic diagram of FIG. 10 shows the use of two wearable target devices 60 for imaging an elbow. As illustrated, two targets devices 60 are disposed on either side of the elbow to be imaged and are displaced with respect to the direction of rotational axis A for CBCT. This arrangement allows at least two data points for calibration of the imaging apparatus, both removed from, but adjacent to, the anatomy of interest. Additional data points can be provided using additional target elements. Target devices 60 can be coupled to each other, such as by a connecting strap that is radio-transparent, or can be separate from each other.

To promote sanitary conditions, (all or a portion of) target device 60 can be disposed within a disposable sheath, such as a plastic disposable film/bag.

Using an embodiment of the present disclosure, calibration of the CBCT system can be achieved using image data from the reconstructed 3D volume. By identifying voxels reconstructed from the target elements or patches 50 of known radio-opaque density, the needed data for calibration can be obtained, allowing an accurate range of HU values to be measured. This allows functions using quantitative image data, such as BMD, to have improved accuracy.

Since the target patches 50 (appearing in the sequence of acquired 2D projection images) are of a known radio-opaque density, the needed data for calibration can be obtained, allowing an accurate range of HU values to be measured.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A wearable calibration target comprising:
a device configured to be worn near a patient's limb; and
one or more calibration patches coupled to the device, each of the one or more calibration patches formed from a material having a known attenuation to X-ray radiation.

2. The wearable calibration target of claim 1 wherein at least one of the calibration patches is formed from water, calcium hydroxyapatite, or potassium phosphate.

3. The wearable calibration target of claim 1 wherein at least one of the calibration patches has a spherical shape.

4. The wearable calibration target of claim 1 wherein the device is a band, and further comprises at least one mounting element to removably attach at least one of the one or more calibration patches to the band.

5. The wearable calibration target of claim 1 wherein the device is a band, and at least one of the one or more calibration patches is woven into or sewed to the band.

6. The wearable calibration target of claim 1 wherein the device is a band, and at least one of the one or more calibration patches is slideably movable to a different position on the band.

7. The wearable calibration target of claim 1 wherein the device is a band having a hook-and-loop fastener.

8. A method comprising:
coupling a flexible calibration device to a limb of a patient, the calibration device including at least one calibration target element coupled to the calibration device, each calibration target element having a known attenuation to X-ray energy;
acquiring a sequence of 2D projection images of the flexible calibration device and the limb on an image detector of a cone beam computed tomography (CBCT) apparatus, wherein the at least one calibration target element appears in the acquired sequence of 2D projection images;
calibrating the image detector of the CBCT apparatus using image data of the calibration target element appearing in the acquired sequence of 2D projection images.

9. The method of claim 8, further comprising, prior to calibrating the image detector, reconstructing at least a portion of a 3D volume image of the limb according to the acquired sequence of 2D projection images;

10. The method of claim 8 wherein coupling the calibration device to the limb comprises:
wrapping the calibration device about the limb of the patient; and
adjusting the calibration device for limb dimensions using a fastener.

11. The method of claim 8 further comprising configuring the at least one calibration target element according to an exam type.

12. A method for calibration of a volume imaging apparatus, comprising:
coupling a first wearable calibration target to an anatomy of a patient, the first calibration target comprising a band having a first radio-opaque calibration patch of a predetermined X-ray radiation attenuation;
using a volume imaging apparatus, acquiring a plurality of 2D projection images of the patient anatomy and the first radio-opaque calibration patch; and
calibrating the volume imaging apparatus according to image data of the first radio-opaque calibration patch from the acquired plurality of 2D projection images.

13. The method of claim 12 further comprising:
coupling a second wearable calibration target to the anatomy of the patient, the second wearable calibration target being spaced from the first calibration target, the second wearable calibration target comprising a band having a second radio-opaque calibration patch of a predetermined X-ray radiation attenuation;
using the volume imaging apparatus, acquiring the plurality of 2D projection images of the patient anatomy and the first and second radio-opaque calibration patches; and
calibrating the volume imaging apparatus according to image data of the first and second radio-opaque calibration patches from the acquired plurality of 2D projection images.

14. The method of claim 13 further comprising coupling the first and second wearable calibration targets such that the patient anatomy is disposed intermediate the first and second wearable calibration targets.

15. The method of claim 12 further comprising:
reconstructing a volume image according to the acquired plurality of 2D projection images; and
calibrating the volume imaging apparatus according to image data from the reconstructed volume image.

16. The method of claim 12 wherein coupling the first wearable calibration target to the patient comprises covering a portion of the anatomy with a portion of the first wearable calibration target.
